(19) United States
(12) Patent Application Publication
(10) Pub. No.: US 2002/0102556 A1
(43) Pub. Date: Aug. 1, 2002

(54) Genotyping by mass spectrometric analysis of short DNA fragments

(76) Inventors: Steven J. Laken, Pepperell, MA (US); Bert Vogelstein, Baltimore, MD (US); Kenneth W. Kinzler, BelAir, MD (US); John D. Groopman, Owings Mills, MD (US); Peta E. Jackson, Baltimore, MD (US); Marlin D. Friesen, Chassieu (FR)

Correspondence Address: BANNER & WITCOFF, 1001 G STREET N W, SUITE 1100, WASHINGTON, DC, 20001, US

(21) Appl. No.: 09/774,021

(22) Filed: Jan. 31, 2001

Related U.S. Application Data
(62) Division of application No. 09/198,340, filed on Nov. 24, 1998, now abandoned.

(51) Int. Cl. C12Q 1/68; C07H 21/04; C12P 19/34

(52) U.S. Cl. 435/6; 435/91.2; 536/24.3

(57) ABSTRACT

Genotyping can be accomplished by analysis of short, defined DNA segments using electrospray ionization mass spectrometry. The DNA segments are produced using specially designed primers to amplify a cDNA or genomic DNA template. The primers contain a recognition site for a restriction endonuclease. The amplification products are digested with the restriction endonuclease. Single nucleotide polymorphisms can be detected rapidly and reliably.

The U.S. Government retains certain rights in this invention due to funding of grant CA43460 awarded by the National Institutes of Health.

### TECHNICAL FIELD OF THE INVENTION

The invention is related to the area of genome analysis. In particular it is related to the field of detection of genetic polymorphisms.

### BACKGROUND OF THE INVENTION

One of the most important results of the revolution in genomics research has been the elucidation of genetic variants associated with specific human diseases. Recent examples include variants in BRCA genes predisposing to breast cancer, a variant in Apo E predisposing to dementia, and a variant in prothrombin predisposing to bleeding disorder (1-3). All of these variations are found at relatively high frequencies in certain populations, and testing for the presence of such mutations can provide critical diagnostic information for management of patients and their families.

The discoveries of such variations have stimulated efforts to design approaches for assessing their presence in DNA from clinical samples. Three factors are particularly important for the success of such efforts: accuracy, throughput, and cost. For the evaluation of an individual (or a few) variants, throughput and cost are not generally limiting, but accuracy remains a continuing concern. Procedures that work well in a research environment are not necessarily appropriate for clinical application, as even a minute fraction of errors in the latter setting can have catastrophic consequences.

Many of the methods currently used for variant analysis employ hybridization with specific oligonucleotide probes that can discriminate between the wild-type and variant sequences. Such hybridizations can occur on filters, chips, gels, or in solution (4). Though generally reliable and useful, hybridization techniques suffer from their qualitative, rather than quantitative, nature; most probes will hybridize to all sequence variants at temperatures slightly below the discrimination optimum. The fact that the extent of hybridization of allele-specific oligonucleotides (ASO) is dependent both on the nature of the variation and the surrounding sequences can make ASO difficult to apply without substantial optimization.

Among the other strategies for genotyping variants, those that employ mass spectrometry (MS) have received particular attention. MS represents an improvement over gel-based and hybridization systems because the mass spectrometer yields precise information on the molecular mass of the analyte, the procedure can be fully automated, and both DNA strands can be analyzed in parallel. MS can directly assess the nature of polymerase chain reaction (PCR) products themselves, while other techniques only indirectly assess such PCR products, either through hybridization probes (as in ASO) or DNA polymerase-generated methods that use PCR products as templates (4). Such indirect methods introduce additional sources of error into the assays.

The feasibility of MS analysis of polymorphic PCR products has been demonstrated (5). However, one limiting factor for analysis of single nucleotide polymorphsims (SNPs) is the mass resolution required for measuring a small difference (9 Da between A and T) in PCR-generated fragments, which are generally on the order of 100 bp long. Procedures have been developed which use PCR products as templates to which peptide nucleic acid probes are hybridized and can then be analyzed by MS (6). This clever technique appears to be useful, but it fails to employ one of the strengths of MS in that the analysis of PCR products is not direct.

There is a continuing need in the art for methods which employ MS but employ direct analysis of amplification products.

### SUMMARY OF THE INVENTION

It is an object of the invention to provide reagents and methods for analyzing genotypes by mass spectrometry. These and other objects of the invention are provided by one or more of the embodiments described below.

One embodiment of the invention provides an isolated primer for amplifying a segment of DNA. The primer comprises a linear oligonucleotide consisting of at least 35 nucleotides. The oligonucleotide comprises a 5′ end and a 3′ end. A first portion of the oligonucleotide consists of at least 13 nucleotides at the 5′ end of the oligonucleotide. A second portion of the oligonucleotide consists of from 5 to 22 nucleotides at the 3′ end of the oligonucleotide. The first and second portions of the oligonucleotide are either precisely complementary or substantially complementary to a first portion and a second portion, respectively, of a segment of a cDNA or genomic DNA. Four to eight nucleotides between the first portion and the second portion of the oligonucleotide comprise a recognition site for a restriction endonuclease that cleaves at least five nucleotides removed from its recognition site. The segment of the cDNA or genomic DNA does not comprise the recognition site.

Another embodiment of the invention provides a pair of purified primers for amplifying a segment of cDNA or genomic DNA. Each primer comprises a linear oligonucleotide consisting of at least 35 nucleotides. A first portion of the oligonucleotide of at least 13 nucleotides at the 5′ end and a second portion of the oligonucleotide of from 5 to 22 nucleotides at the 3′ end are either precisely complementary or substantially complementary to a first portion and a second portion of a cDNA or genomic DNA. Between the first portion and the second portion of the oligonucleotide are 4-8 nucleotides which comprise a recognition site for a restriction endonuclease that cleaves at least five nucleotides from its recognition site. The segment of the cDNA or genomic DNA does not comprise the recognition site for the restriction endonuclease. Each primer of the pair of primers is complementary to an opposite strand of a double stranded cDNA or genomic DNA molecule. The pair of primers is complementary to two non-contiguous portions of the double stranded cDNA or genomic DNA molecule, such that 1 to 20 nucleotides separate the two non-contiguous portions of the double stranded cDNA or genomic DNA molecule.

Still another embodiment of the invention provides a kit comprising a plurality of pairs of primers as described in the preceding paragraph.

Yet another embodiment of the invention provides a method for producing a short segment of DNA, suitable for analysis by MS. The method comprises the steps of amplifying cDNA or genomic DNA using the pair of primers described above to form amplified DNA and digesting the amplified DNA with the restriction endonuclease to form a short segment of DNA.

A further embodiment of the invention provides a method for analyzing a first short segment of DNA comprising a first polymorphic nucleotide to distinguish the first short segment of DNA from a second short segment of DNA comprising a second polymorphic nucleotide. The method comprises the step of applying a mixture of DNA segments to an electrospray ionization/mass spectrometer, whereby the DNA segments are denatured and the denatured segments are separated. The mixture of DNA segments is made by the process of amplifying cDNA or genomic DNA of a subject using the pair of primers described above to form amplified DNA and digesting the amplified DNA with the restriction endonuclease to form a short segment of DNA.

The invention thus provides the art with novel tools and methods for analyzing the genotype of living organisms, including humans, by electrospray ionization mass spectrometry.

### BRIEF DESCRIPTION OF THE DRAWINGS

FIG. 1 displays the general strategy for the preparation of DNA suitable for short oligonucleotide mass analysis (SOMA). A template is PCR-amplified with primers containing an artificial BpmI restriction endonuclease sequence (CTGGAG) embedded within sequences perfectly complementary to the genomic region of interest. The PCR product is digested with BpmI, and the internal (interrogated) sequence released by BpmI digestion is analysed by the mass spectrometer.

FIGS. 2A, 2B, and 2C illustrate full-scan electrospray mass spectra of 15-mer oligonucleotide standards corresponding to the antisense strands of the APC codon 1307 AAA allele (FIG. 2A) and ATA allele FIG. 2B) . The mass that is the most amenable to detection by the mass spectrometer is the [M-3H]³⁻0 peak corresponding to a m/z of 1519.3 and 1522.3 for the AAA and ATA alleles, respectively. FIG. 2C shows the electrospray mass spectrum for the simultaneous ESI-MS analysis of these two oligonucleotide standards, showing baseline separation for the two [M-3H]³⁻ ions.

FIGS. 3A and 3B demonstrate ESI-MS analysis of APC codon 1307 variants. The four mass chromatograms for each patient represent the AAA sense (s) mass, the AAA antisense (as) mass, the ATA sense (s) mass and the ATA antisense (as) mass, respectively. The patient in FIG. 3A has the ATA/ATA homozygous genotype, while that in FIG. 3B has the ATA/AAA heterozygous genotype.

FIGS. 4A, 4B, and 4C demonstrate ESI-MS/MS SRM analysis of APC codon 1493 variants. Mass chromatograms obtained from genomic DNA of patients with the ACG/ACA, ACA/ACA, and ACG/ACG genotypes, respectively, are presented in FIG. 4A, FIG. 4B, and FIG. 4C, respectively. The masses representing the sense (s) and antisense (as) BpmI fragments corresponding to the variant sequences are indicated.

FIGS. 5A and 5B demonstrate simultaneous analysis of three different APC variants for two patients. For each patient, PCR products containing APC codons 486, 545, and 1756 were combined and introduced into the mass spectrometer via the HPLC. The sense (s) and antisense (as) signals are indicated for each genotype. FIG. 5A represents an individual homozygous at each of the analyzed codons, and FIG. 5B was from an individual homozygous for the other allele at each of these codons.

### DETAILED DESCRIPTION OF THE INVENTION

The present invention provides a method of genotype analysis in which short, defined fragments of amplification products are produced by simple enzymatic digestion and directly analyzed by electro-spray ionization mass spectrometry (ESI-MS). The method, called SOMA (Short Oligonucleotide Mass Analysis), is simple to implement, extremely accurate, and applicable to most DNA variations.

The SOMA technique utilizes short DNA segments of defined length. The segments are produced by amplification of a segment of cDNA or genomic DNA of approximately 50, 60, 70, 80, 90, 100, 110, 120, 130, 140, or 150 bp, preferably about 100 bp, using specially designed amplification primers. The cDNA or genomic DNA can be isolated from a subject organism by methods known in the art. The subject organism can be any organism, for example a human or other animal, a plant, a fungus, or a microorganism such as a bacterium or a virus.

Primers can be either precisely complementary or substantially complementary to two non-contiguous portions of the segment of cDNA or genomic DNA. The term “precisely complementary” as used herein refers to nucleic acids that are complementary at every base pair. Thus, a primer is precisely complementary to its template sequence if every nucleotide of the primer is complementary to every corresponding nucleotide of the template sequence. The term “substantially complementary” refers to nucleotide sequences which are at least 90% identical to their corresponding template sequences as determined by the Smith-Waterman homology search algorithm as implemented in the MPSRCH program (Oxford Molecular) using an affine gap search with a gap open penalty of 12 and a gap extension penalty of 1.

The two non-contiguous portions of the cDNA or genomic DNA to which the primers are complementary flank the portion of the cDNA or genomic DNA containing the polymorphism. The two non-contiguous portions are separated from each other by 1 to about 10, 12, 14, 16, 18, 20, 22, 24, 26, 28, 30, 35, or 40 bp, and preferably by 1 to 20 bp. The two primers are complementary to opposite strands of the cDNA or genomic DNA, such that amplification produces a segment of cDNA or genomic DNA which contains the polymorphism to be analyzed flanked by the primer sequences.

The primer can be a linear oligonucleotide comprising at least 20, 25, 30, 35, 40, 45, 50, 60, or 70 nucleotides, preferably comprising at least 35 nucleotides, and more preferably consisting of from 41 to 44 nucleotides. The primer can comprise a first portion at its 5′ end, which comprises at least 8, 9, 10, 11, 12, 13, 14, 15, 16, 18, 20, 22, or 25 nucleotides. Preferably the first portion comprises at least 13 nucleotides. More preferably the first portion consists of from 21 to 22 nucleotides. The first portion of the primer is complementary, or substantially so, to one strand of the cDNA or genomic DNA segment. The primer can also comprise a second portion at its 3′ end, which consists of at least 3, 4, 5, 6, 7, 8, or 10 to 18, 19, 20, 21, 22, 23, 24, 26, 28, or 30 nucleotides Preferably the second portion consists of from 5 to 22 nucleotides, and more preferably the second portion consists of from 14 to 16 nucleotides. The second portion of the primer is complementary, or substantially so, to a second portion of the same strand of the cDNA or genomic DNA segment to which the first primer portion is complementary.

The first and second portions of the primer are separated by a sequence consisting of from 3, 4, or 5 to 7, 8, 9, or 10 nucleotides. Preferably the separating sequence consists of from 4 to 8 nucleotides. The separating sequence comprises a restriction endonuclease recognition sequence. A “restriction endonuclease” or “restriction enzyme” is a bacterial enzyme that binds to a specific recognition site on a double stranded DNA molecule and cleaves the molecule at a specific cleavage site. The “recognition site” is a nucleotide sequence within the double stranded DNA molecule to which the endonuclease binds. The “cleavage site” is the position at which the endonuclease cuts the double stranded DNA molecule. The position of the cleavage site is relative to the recognition site and is a characteristic of the endonuclease.

The restriction endonuclease whose recognition sequence is used is a restriction endonuclease that cleaves at a site distinct from the recognition sequence. The restriction endonuclease can be, for example, a Type IIS restriction endonuclease such as BpmI, BsgI, BseRI or BciVI. Type IIS restriction endonucleases have asymmetric recognition sites and cleave at a specific distance of up to 20 bp outside their recognition site (20). Using a restriction endonuclease that cleaves outside the primer is advantageous, because the product of endonuclease treatment can then be a smaller DNA segment than if the endonuclease cleaved within the primer, and a smaller DNA segment enhances the sensitivity of the method. The restriction endonuclease should have a cleavage site distal from its recongnition site by at least 3, 4, 5, 6, 8, 10, 12, or 15 nucleotides, and preferably by at least 8 nucleotides. Preferably, the restriction endonuclease recognition sequence will not be found within the amplified segment of cDNA or genomic DNA.

The two portions of the cDNA or genomic DNA which are complementary to the first and second portions of the primer can be separated by from 0, 1, 2, 3, or 4 nucleotides to 8, 9, 10, 11, 12, 13, 14, 15, or 16 nucleotides. Preferably they are separated by from 4 to 8 nucleotides and more preferably they are separated by 6 nucleotides.

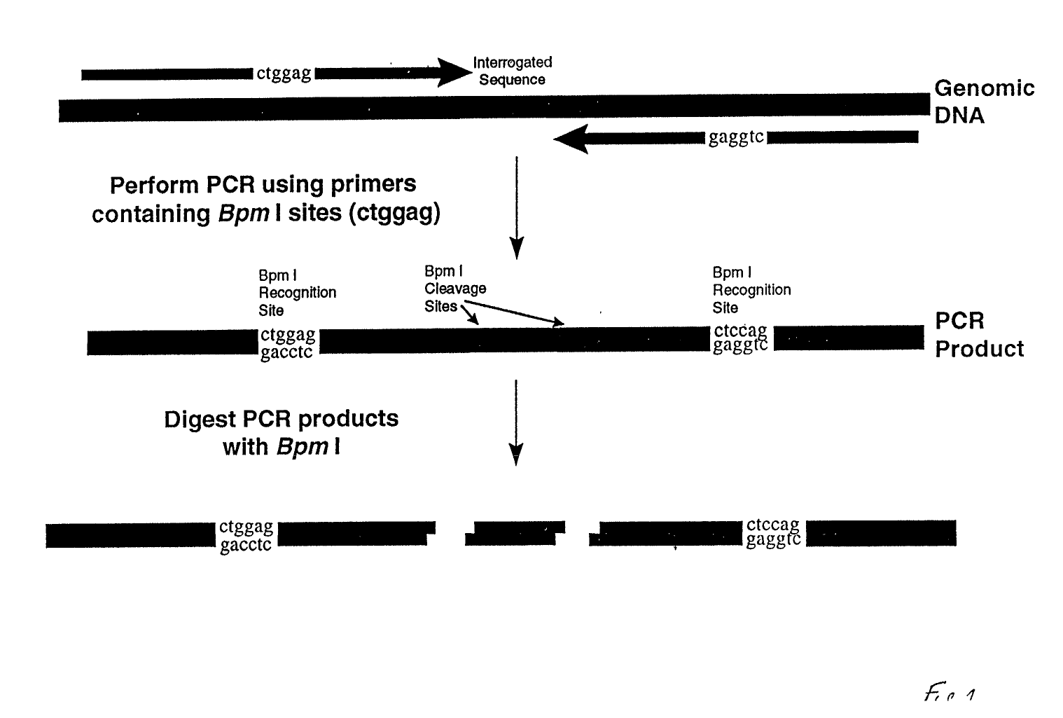

A pair of such primers as described above which flank a segment of cDNA or genomic DNA containing a polymorphism can be used to amplify the polymorphism. Each primer of the pair is complementary to a different strand of the cDNA or genomic DNA. Therefore, if a first primer of a pair is complementary to the coding strand of the cDNA or genomic DNA segment, then the other primer of the pair must be complementary to the non-coding strand, i.e., the opposite strand, of the cDNA or genomic DNA segment to be amplified. In this way, when amplification is performed using the pair of primers, the resulting amplified DNA will contain a copy of the segment of cDNA or genomic DNA between the portions complementary to the primers (FIG. 1).

The region of cDNA or genomic DNA containing the polymorphism between the primer-complementary portions can vary in length from 1, 2, 3, 4, or 5 bp to about 16, 18, 20, 22, 24, 26, 30, 35, or 40 bp, but preferably is in the range from 1 to 20 bp. The length of this region is determined by several factors relating to the design of the primer pair used for amplification. Those factors include the composition and length of the portions of cDNA or genomic DNA to which the primers are complementary and the distance between the recognition and cleavage sites of the restriction endonuclease. Generally, use of shorter segments of cDNA or genomic DNA yield greater mass resolution and greater sensitivity.

Primers according to the invention can be synthesized by any method known in the art for oligonucleotide synthesis. For example, solid phase oligonucleotide synthesis can be performed by sequentially linking 5′ blocked nucleotides to a nascent oligonucleotide attached to a resin, followed by oxidizing and unblocking to form phosphate diester linkages (21). Primers according to the invention are isolated. The term “isolated” as used herein refers to a molecule that is substantially free of undesired contaminants, such as molecules having other sequences.

Primers of the invention can be made available as a kit. A kit contains, in one or more divided or undivided vessels, a plurality of primers for use in analyzing one or more specific polymorphisms. The primers in a kit are designed to be used together, for example, in pairs which are complementary to regions of a cDNA or genomic DNA which flank a particular polymorphism. A kit can optionally contain the restriction endonuclease whose recognition sequence is contained in the primers. A kit can also contain several primers or several pairs of primers for use in genotyping at least two related or unrelated polymorphisms.

To carry out genotyping according to the invention, the primers are used to amplify a segment from a sample of template cDNA or genomic DNA. The term “amplification” as used herein refers to any process using a pair of primers described above that produces multiple copies (ng amounts) of the segment of cDNA or genomic DNA between and including the portion complementary to the 5′ ends of the pair of primers. The process of amplification can be carried out, for example, using the polymerase chain reaction (PCR) technique (see, e.g. U.S. Pat. No. 4,683,195 or reference 18) or by any other amplification method known in the art.

The amplified product can be cleaved using the restriction endonuclease whose recognition site is present in the primers. When the enzyme cleaves the DNA, it breaks a covalent bond at a discrete location on each strand. Digestion of a double stranded DNA molecule with a restriction endonuclease refers to the process of allowing the endonuclease to bind to its recognition site, cleave at its cleavage site, and release the cleaved DNA products. Because each member of the pair of primers of this invention contains a recognition site for the restriction endonuclease, digestion of the amplified product with the endonuclease will result in cleavage at two sites and consequently the release of a defined fragment or segment of the product. The product of the restriction endonuclease digestion will be a short, defined segment of double stranded DNA, whose length can be from 1, 2, 3, 4, 5, 6, 7, 8, 9, or 10 to about 16, 18, 20, 22, 24, 26, 30, 35,or 40 bp, but preferably is from about 7 to about 20 bp. The appropriate length of this segment is determined by the resolution of the MS method used for mass analysis. If the segment is too long, the analysis may be less sensitive.

The DNA cleavage product can be analyzed directly by ESI-MS or following an optional purification step. Purification can be carried out, for example, by reverse phase HPLC. The term “denature” refers to the dissociation of a double stranded DNA molecule to yield two single stranded DNA molecules. The “separation” of DNA molecules by ESI-MS refers to their physical separation from other molecules based on mass/charge ratio. The analysis can also be automated, for example, by performing the amplification and digestion steps in microtiter plates at a robotic workstation and loading the samples via an autosampler into an ESI-MS instrument. Loading on an HPLC can also be automated prior to ESI-MS. This would permit the rapid and sequential analysis of a large number of polymorphic fragments, for example, obtained from a number of patients to be screened.

The above disclosure generally describes the present invention. A more complete understanding can be obtained by reference to the following specific examples, which are provided herein for purposes of illustration only and are not intended to limit the scope of the invention. Examples 2-4 present details of the ESI-MS analysis using polymorphisms of the human adenomatous polyposis carcinoma (APC) gene.

### EXAMPLE 1

Generation of Short DNA Segments for ESI-MS

In order to unambiguously differentiate DNA fragments using a 2000 Da ion-trap mass spectrometer, it was first necessary to generate short, specific PCR products from complex genomes. To produce such short fragments (<20 bases), PCR amplification was carried out with primers containing a sequence for the type II restriction enzyme, BpmI (FIG. 1).

Primers of 41-44 bases in length were designed so that 21-22 bases at the 5′ end and 14-16 bases at the 3′ end were precisely complementary to a 41-44 genomic sequence. The six base BpmI recognition sequence was placed between the 21-22 and 14-16 base portions, precisely replacing the 6 bases that were normally present at this position in the genome (FIG. 1). Each PCR-primer contained at least 35 bases complementary to a specific genomic sequence, and the PCR fragments generated were only ˜100 bp in length, thus ensuring that the PCR reaction was very robust.

PCR was performed as described (18). Reactions were performed with 25-50 ng of human genomic DNA, in 50 μl. Thermal cycling conditions were 95° C. for 2 min, followed by 40 cycles of 95° C. for 30 sec., 60° C. for 30 sec., and 72° C. for 30 sec.

Following PCR amplification, low molecular weight oligonucleotides were obtained for mass analysis by restriction endonuclease digestion. 12 μl of the PCR product were digested with 10 units BpmI for 2 hours at 37° C. in 50 μl. One unit of restriction endonuclease activity is the amount of enzyme required to completely digest 1 μg of substrate DNA in a 50 μl reaction in one hour at 37° C. DNA was extracted using one volume phenol/chloroform and precipitated in the presence of 3-5 μl of SeeDNA (Amersham), 6 volumes ethanol, and one third volume of 7.5 M ammonium acetate. After washing the pellets with 70% ethanol, the samples were allowed to air dry and resuspended in 10 μl of a solution of aqueous 0.4 M 1,1,1,3,3,3-hexafluoro-2-propanol (HFIP, Sigma) and methanol (85:15, v/v), of which 5 μl was typically injected for ESI-MS analysis.

After PCR amplification and BpmI digestion, DNA was purified by standard phenol/chloroform extraction and ethanol precipitation. It was not necessary to separate the larger (>40 bases) end fragments produced by BpmI digestion, as these were not confused with the short (7-20 bases), internal, variant fragments to be queried. Under ESI-MS conditions, these internal DNA fragments denatured and separated to produce detectable masses representing both the sense and antisense strands.

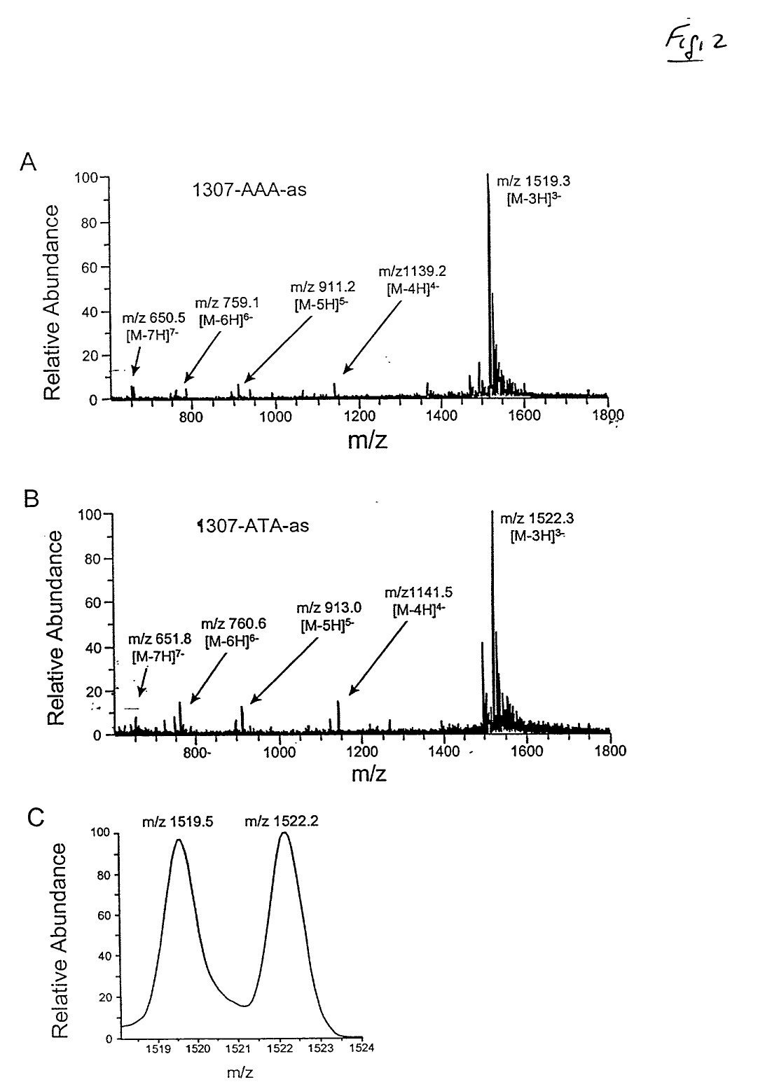

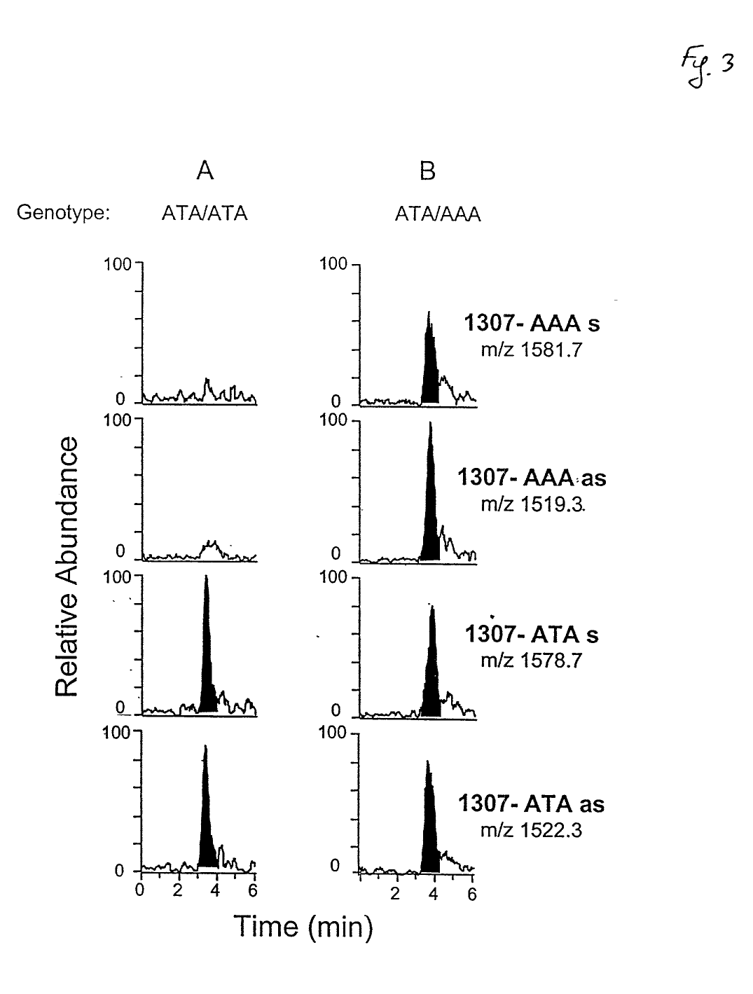

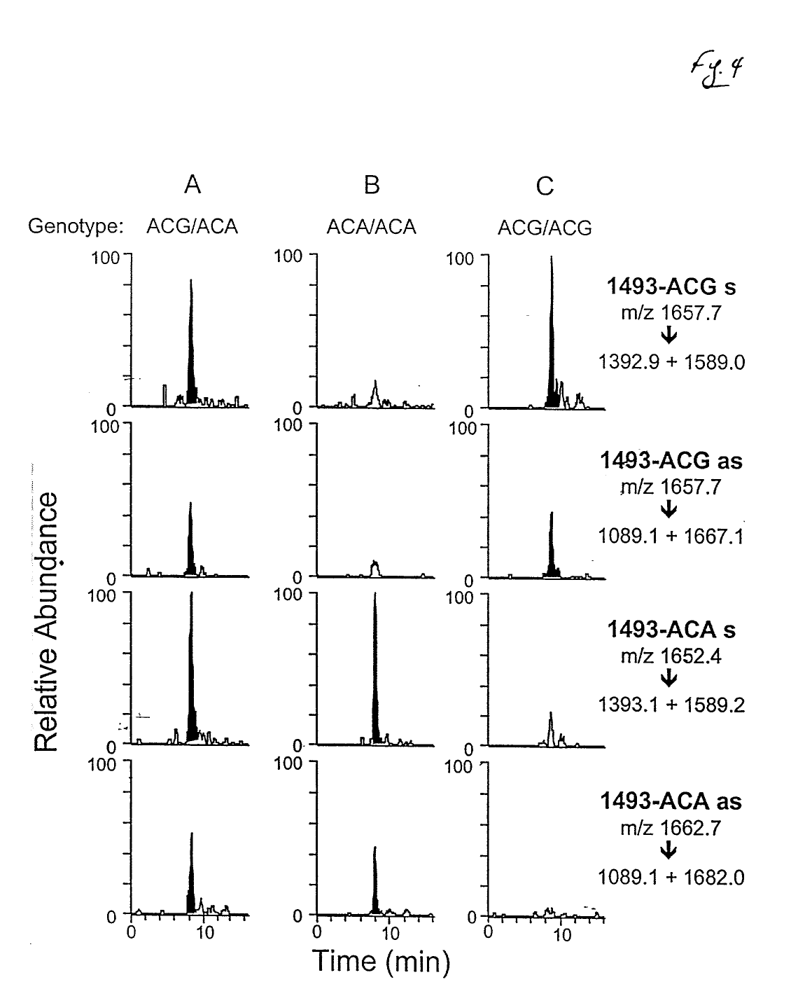

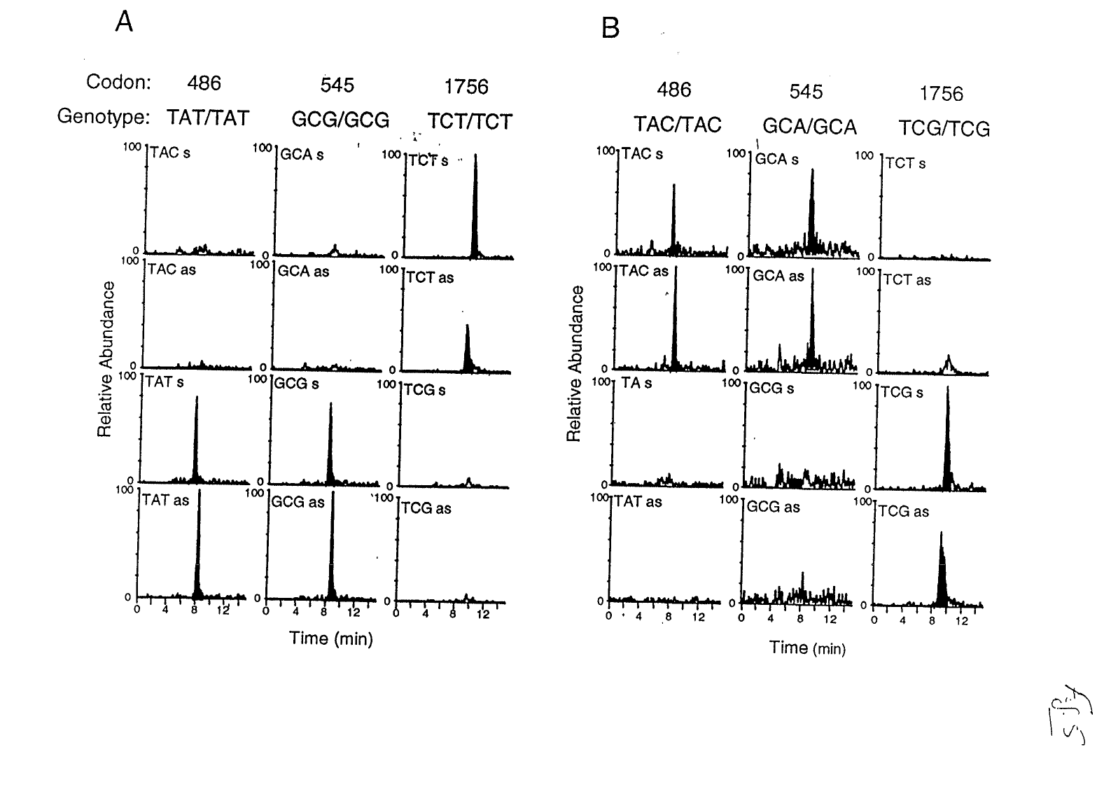

Oligonucleotide fragments for MS analysis were purified by reverse phase HPLC. Introduction of oligonucleotides into the HPLC coupled to the mass spectrometer was carried out at ˜18 μl/min on a 15 cm×800 μm I.D Vydac C-18 reverse phase column (5 μm, 300 Å pore size, LC Packings, Amsterdam, NL). To obtain this flow rate, Waters 515 HPLC pumps (Waters Corp., Milford, Mass., USA) operating at 0.2 ml/min were connected to an LC Packings Accurate microflow splitter. HPLC solvents were prepared from a stock solution of aqueous 0.8 M HFIP, adjusted to pH 7.0 with triethylamine, then diluted to 0.4 M (with water for solvent A and methanol for solvent B, as described by Apffel (19)). Initial analysis was carried out isocratically with a 20% A/80% B solvent mixture (see, for example, FIG. 2 and FIG. 3). Alternatively, an initial solvent concentration of 70% A/30% B was programmed to 50% A/50% B after 1 minute, where it was held for 10 minutes (see FIG. 4 and FIG. 5). The majority of potentially interfering compounds not removed by phenol/chloroform extraction and ethanol precipitation eluted with the void volume and were diverted to waste, while oligonucleotides of interest were eluted as the methanol concentration was increased from 15% to 25%.

### EXAMPLE 2

ESI-MS Analysis of the APC gene codon 1307 variant

This variant (I1307K) is present in 6% of Ashkenazi Jews, and is associated with a ˜2-fold increase in colorectal cancer risk (7). The wild-type and variant sequences differ only at codon 1307 (ATA vs. AAA), and the A to T mutation represents the most difficult one to detect by MS analysis because the A to T change reflects only a 9 Da difference in mass.

Mass spectra were obtained on an LCQ ion-trap mass spectrometer (Finnigan MAT, San Jose, Calif., USA) equipped with an electrospray ionization source operated in the negative ionization mode. To increase sensitivity, a 33 gauge stainless steel ESI needle, covered with 1/16 Teflon tubing outside the ESI source for insulation from the high voltage, was used in place of the standard fused silica ESI needle. The instrument was tuned daily by infusion at 1 μI/min of one of the oligonucleotides studied (10 ng/μl in 70% A/30% B) into the 18 μl/min HPLC mobile phase through a low dead-volume tee. Typical settings for the spray voltage were −2.5 to −5 kV. The stainless steel heated capillary temperature was held at 180° C.

Primers were designed according to the strategy outlined in FIG. 1, so that 15-mer oligonucleotides were generated following BpmI digestion. Primers used for PCR amplification of the APC variants were: 1307 sense (SEQ ID NO:1), 5′-AGACGACACAGGAAGCAGATTCTGGAGATACCCTGCAAATAGC-3; and 1307 antisense (SEQ ID NO:2), 5′-GGAACTTCGCTCACAGGATCTTCTGGAGACCTAGTTCCAATC-3′. The expected sizes of the product was 100 bp. Synthetically-generated antisense oligonucleotides, corresponding to two of the four expected fragments, were used to optimize the ESI-MS analysis. For both compounds, the most intense ions observed were [M-3H]³⁻ ions at m/z (mass to charge) 1519.3 (AAA) and m/z 1522.3 (ATA) (FIG. 2A and FIG. 2B). The difference in m/z between these [M-3H]³⁻ ions is 3 Da, which was easily resolved under the experimental conditions (FIG. 2C).

For detection of the I1307K variants (FIGS. 3A and 3B), the mass spectrometer was programmed to acquire data in the profile mode (1 μscan; 1000 msec; isolation width 2.0 Da) using two scan events monitoring two [M-3H]³⁻ ions simultaneously. Scan event 1: m/z 1581.7 [5′-pAGAAAAAAAAGAAAA-3′, SEQ ID NO:3], 1519.3 [5′-pTTCTTTTTTTTCTGC-3′, SEQ ID NO:4]. Scan event 2: m/z 1578.7 [5′-pAGAAATAAAAGAAAA-3′, SEQ ID NO:5], 1522.3 [5′-pTTCTTTTATTTCTGC-3′, SEQ ID NO:6]. Reconstructed ion chromatograms were generated and smoothed from this raw data using an isolation width of 1.0 Da and normalized to the largest of the four oligonucleotide ion peaks.

Genomic DNA was used as a template for PCR, and the PCR products digested with BpmI and purified by phenol/chloroform extraction. The samples were introduced into the mass spectrometer using the HPLC and [M-3H]³⁻ ion masses characteristic of the two sense (m/z 1581.7 and 1587.7) and two antisense strands (m/z 1519.3 and 1522.3) were measured by selected ion monitoring as a function of time. It was found that there was sufficient material generated from the digestion of ¼ of a 50 μl PCR reaction for two ESI-MS injections. Furthermore, the simple phenol-chloroform purification was sufficient to obtain good mass chromatographic peaks with minimal interference from other compounds in the channels monitored (FIG. 3). Sixteen human samples, which had previously been analysed by sequencing, were genotyped with this method. Samples from subjects who were heterozygous had peaks in all four channels monitored (i.e., had both the wild-type and mutant sense and antisense strands), whereas samples from individuals who were homozygous for the wild-type allele only had peaks in the two wild-type channels. There was 100% concordance between SOMA and sequencing results.

### EXAMPLE 3

ESI-MS Analysis of the APC Codon 1493 Variant

A second variant in the APC gene (ACA or ACG at codon 1493) was selected to demonstrate the general applicability of the methodology, even in difficult cases. This variant is not associated with disease, but is a common polymorphism which can be used for linkage analysis in families with familial adenomatous polyposis (8).

Primers used for PCR amplification of the APC variants were: 1493 sense, 5′-TTCAGAGGGTCCAGGTTCTTCCTGGAGCTGATACTTTATTACA-3′ (SEQ ID NO:7); and 1493 antisense, 5′GCACTCAGGCTGGATGAACAACTGGAGCCATCTGGAGTACT-3′ (SEQ ID NO:8). The expected size of the product was 100 bp. The internal fragments generated by SOMA were designed to be 16 bp long. Moreover, for one of the alleles (ACG), the sense (5′-pTTTTGCCACGGAAAGT-3′, SEQ ID NO:9) and antisense (5′-pTTTCCGTGGCAAAATG-3′, SEQ ID NO:10) oligonucleotides had different base sequences but the same mass. This resulted in two oligonucleotide [M-3H]³⁻ ions with identical mass-to-charge ratios at 1657.7 which could not be resolved by ESI-MS. However, it was found that ESI-MS/MS selected reaction monitoring could easily differentiate between the four oligonucleotide ions. Heterozygotes were identified by the presence of chromatographic peaks in all four channels, while peaks in the sense and antisense channels of one allele indicated a homozygous sample (FIG. 4). Of 50 individuals genotyped at codon 1493, there was a 100% correlation between the results obtained by SOMA and sequencing.

Although it might be expected that the four chromatographic peaks obtained for the four oligonucleotides produced from a heterozygote would be of equal intensity, this is not always the case. Oligonucleotide base sequence, length, and conformation cause variations in ESI-MS response factors. However, for all variants we studied, the relative response factors measured for synthetic oligonucleotide standards closely approximated those measured for the four oligonucleotides generated from human DNA. This allowed straightforward normalization of the signals obtained if desired, though no normalization was used in the data presented in FIGS. 2-5. Interference from oligonucleotide-cation adducts and non-specific DNA fragments produced background signals for certain variants. This background could be reduced with improved chromatographic separation and sample cleanup, or by simply redesigning the primers for amplification to produce a slightly different internal fragment containing the sequence variation. MS/MS is also a powerful technique for improving selectivity, even in the presence of interfering compounds (FIG. 4). To date, SOMA has been used to analyze seven different single nucleotide variations. Of these, all four species (sense and antisense from the two alleles) could be readily discerned in five cases on the first try, while in two cases, different primers, producing a slightly different length or position of interrogated sequence, had to be designed to produce acceptable results.

### EXAMPLE 4

Simultaneous Analysis of Multiple Variants

Three common polymorphisms in the APC gene, at codons 485, 545, and 1756 (8), were chosen to demonstrate that multiple polymorphisms could be analysed in parallel by ESI-MS.

For detection of multiple variants (FIG. 5), the mass spectrometer was programmed to acquire data in the profile mode (1 μscan; 30 msec; isolation width 3.0 Da) using two ˜1.4-sec scan events monitoring 16 [M-2H]²⁻ ions simultaneously. (Scan event 1: 486-TAC-s m/z 1271.8 [5′-pTGTACGGG-3′]; 486-TAC-as m/z 1231.3 [5′-pCGTACATT-3′]; 545-GCA-s m/z 1407.9 [5′-pATTGCAAGT-3′]; 545-GCA-as m/z 1399.9 [5′-pTTGCAATAA-3′]; 1756-TCG-s m/z 1688.6 [5′-pGCGTCGTCTTC-3′, SEQ ID NO:11]; 1756-TCG-as m/z 1726.6 [5′-pAGACGACGCAG-3′, SEQ ID NO:12]. Scan event 2: 486-TAT-s m/z 1279.3 [5′-pTGTATGGG-3′]; 486-TAT-as m/z 1223.3 [5′-pCATACATT-3′]; 545-GCG-s m/z 1415.9 [5′-pATTGCGAGT-3′]; 545-GCG-as m/z 1392.4 [5′-pTCGCAATAA-3′]; 1756-TCT-s m/z 1676.1 [5′-pGCGTCTTCTTC3′, SEQ ID NO:13]; 1756-TCT-as m/z 1738.6 [5′-pAGAAGACGCAG-3′, SEQ ID NO:14]). Reconstructed ion chromatograms were generated and smoothed from this raw data using an isolation width of 1.0 Da and normalized to the largest of the four oligonucleotide ion peaks for each variant.

For analysis of DNA segments that have identical masses but different nucleotide sequences, the technique of tandem MS (MS/MS) was applied to distinguish the segments. ESI-MS/MS was used for analysis of the 1493 variant (FIG. 4). Using this technique, the four oligonucleotide ions studied were isolated in the ion-trap and subjected to collisional-induced dissociation at 60% collision energy, resulting in sequence-specific fragment ions of the four original ions. Signals from two MS/MS fragment ions were summed as a function of time for each of the four oligonucleotide [M-3H]³⁻ ions monitored. The mass spectrometer was programmed to acquire data in the profile mode (1 μscan; 500 msec; isolation width 3.5 Da) using four scan events monitoring each [M-2H]²⁻ oligonucleotide ion individually. (Scan event 1: ACG-s: m/z 1657.7->1392.9+1589.0. Scan event 2: ACG-as: m/z 1657.7->1089.1+1667.1. Scan event 3: ACA-s: m/z 1652.4->1393.1+1589.2. Scan event 4: ACA-as: m/z 1662.7->1089.1+1682.0.) Reconstructed ion chromatograms were generated and smoothed from this raw data using an isolation width of 1.0 Da and normalized to the largest of the four oligonucleotide ion peaks.

Primers used for PCR amplification of the variants were: 486 sense,

| | |
|---|---|
| 5′-GGACTACAGGCCATTGCAGAACTGGAGCAAGTGGACTGTGAAA-3′ | (SEQ ID NO:15); 486 antisense, |
| 5′-AGCATATCGTCTTAGTGTAATACTGGAGTGGTCATTAGTAAG-3′ | (SEQ ID NO:16); 545 sense, |
| 5′-ATTTTATGTATAAATTAATCTCTGGAGGATTAATTTGCAGGTT-3′ | (SEQ ID NO:17); 545 antisense, |
| 5′-TTTACTATTTACATCTGCTCGCCTGGAGAAATTCCTCAAAAC-3′ | (SEQ ID NO:18); 1756 sense, |
| 5′-TTTCCGTGTGAAAAAGATAATCTGGAGGGTCCAGCAAGCATCT-3′ | (SEQ ID NO:19); and 1756 antisense, |
| 5′-GGTTTCTTTTTCTTACCATCTACTGGAGTTTTGTTGGGTGCA-3′ | (SEQ ID NO:20). |

The expected sizes of the products were 93 bp for codon 486, 94 bp for codon 545, and 96 bp for codon 1756. Regions around each of the polymorphic sites were amplified in separate PCR reactions and BpmI digestion was performed, producing DNA fragments of 8, 9, and 11 bases containing codons 485, 545, and 1756, respectively. The three reaction mixtures from each individual were then combined, purified by phenol-chloroform extraction, and introduced into the mass spectrometer using the HPLC. Twelve [M-2H]²⁻ ion masses, characteristic of the three polymorphisms, were monitored by ESI-MS selected ion monitoring. Results for simultaneous determination of polymorphisms at the three codons in two individuals homozygous for each polymorphism are shown in FIG. 5. Heterozygotes displayed the expected four peaks (not shown). The results obtained by SOMA and sequencing were again perfectly concordant.

Prior methods of identifying sequence variations in human DNA by MS have for the most part employed matrix-assisted laser desorption/ionisation time-of-flight mass spectrometry (MALDI-TOF). With that technique, a UV laser pulse to the sample in a fixed matrix causes ionized biomolecules to be released into the gas phase where they can be extracted for mass separation. MALDI-TOF has been used most successfully to analyze variations which are characterized by large mass differences (5, 11, 12). When used to identify SNPs, the use of MALDI-TOF has usually required hybridization of small fragments to PCR-amplified DNA for adequate resolution (6, 13-16). In addition, use of the technique has been hampered by interference from sodium and potassium adduct ions, which can lead to errors in the determination of ion mass and decreased signal-to-noise ratios.

Although PCR has previously been coupled with ESI-MS to assess insertion/deletion-type variations in human DNA (17), this invention represents the first application of ESI-MS to detect SNPs. The ESI mass spectrum gives information on both alleles and for both sense and antisense strands. The approach is applicable to any subtle variation and can measure the variations with the smallest possible mass difference with excellent resolution. The method requires just picomole quantities of oligonucleotide for each analysis. Sample clean-up, involving standard phenol/chloroform extraction and ethanol precipitation, is simple, quick and amenable to automation.

References

1. Struewing, J. P., Hartge, P., Wacholder, S., Baker, S. M., Berlin, M., McAdams, M., et al. 1997. The risk of cancer associated with specific mutations of BRCA1and BRCA2 among Ashkenazi Jews. N. Engl. J. Med. 336:1401-1408

2. Martinez, M., Campion, D., Brice, A., Hannequin, D., Dubois, B., Didierjean, O., et al. 1998. Apolipoprotein E epsilon4 allele and familial aggregation of Alzheimer disease. Arch. Neurol. 55:810-816.

3. De Stefano, V., Chiusolo, P., Paciaroni, K., Casorelli, I., Rossi E., Molinari, M. et al. 1998. Prothrombin G20210A mutant genotype is a risk factor for cerebrovascular ischemic disease in young patients. Blood 91:3562-3565.

4. Nollau, P. and Wagener, C. 1997. Methods for detection of point mutations: performance and quality assessment. IFCC Scientific Division, Committee on Molecular Biology Techniques. Clin. Chem. 43:1114-1128.

5. Ross, P. L. and Belgrader, P. 1997. Analysis of short tandem repeat polymorphisms in human DNA by matrix- assisted laser desorption/ionization mass spectrometry. Anal. Chem. 69:3966-3972.

6. Griffin, T. J., Tang, W. and Smith, L. M. 1997. Genetic analysis by peptide nucleic acid affinity MALDI-TOF mass spectrometry. Nat. Biotechnol. 15:1368-1372.

7. Laken, S. J., Petersen, G. M., Gruber, S. B., Oddoux, C., Ostrer, H., Giardiello, F. M. et al. 1997. Familial colorectal cancer in Ashkenazim due to a hypermutable tract in APC. Nat. Genet. 17:79-83.

8. Powell, S. M., Zilz, N., Beazer-Barclay, Y., Bryan, T. M., Hamilton, S. R., Thibodeau, S. N. et al. 1992. APC mutations occur early during colorectal tumorigenesis. Nature 359:235-237.

9. Miketova, P. and Schram, K. H. 1997. Mass spectrometry of nucleotides and oligonucleotides. Mol. Biotechnol. 8:249-253.

10. Crain, P. F. and McCloskey, J. A. 1998. Applications of mass spectrometry to the characterization of oligonucleotides and nucleic acids. Curr. Opin. Biotechnol. 9:25-34.

11. Braun, A., Little, D. P., Reuter, D., Muller-Mysok, B. and Koster, H. 1997. Improved analysis of microsatellites using mass spectrometry. Genomics 46:18-23.

12. Wada, Y. 1998. Separate analysis of complementary strands of restriction enzyme- digested DNA. An application of restriction fragment mass mapping by matrix-assisted laser desorption/ionization mass spectrometry. J. Mass. Spectrom. 33:187-192.

13. Ross, P. L., Lee, K. and Belgrader, P. 1997. Discrimination of single-nucleotide polymorphisms in human DNA using peptide nucleic acid probes detected by MALDI-TOF mass spectrometry. Anal. Chem. 69:4197-202.

14. Fei, Z., Ono, T. and Smith, L. M. 1998. MALDI-TOF mass spectrometric typing of single nucleotide polymorphisms with mass-tagged ddNTPs. Nucleic Acids Res. 26:2827-2828.

15. Higgins, G. S., Little, D. P. and Koster, H. 1997. Competitive oligonucleotide single-base extension combined with mass spectrometric detection for mutation screening. Biotechniques 23:710-714.

16. Haff, L.A. and Smirnov, I.P. 1997. Single-nucleotide polymorphism identification assays using a thermostable DNA polymerase and delayed extraction MALDI-TOF mass spectrometry. Genome Res. 7:378-388.

17. Naito, Y., Ishikawa, K., Koga, Y., Tsuneyoshi, T., Terunuma, H., and Arakawa, R. 1997. Genetic Diagnosis by Polymerase Chain Reaction and Electrospray Ionization Mass Spectrometry: Detection of Five Base Deletion From Blood DNA of a Familial Adenomatous Polyposis Patient. J. Am. Soc. Mass Spectrom. 8:737-742.

18. Riggins, G. J., Kinzler, K. W., Vogelstein, B. and Thiagalingam, S. 1997. Frequency of Smad gene mutations in human cancers. Cancer Res. 57:2578-2580.

19. Apffel, A., Chakel, J. A., Fischer, S., Lichtenwalter, K. and Hancock, W. S. 1997. Analysis of Oligonucleotides by HPLC-Electrospray Ionization Mass Spectrometry. Anal. Chem. 69:1320-1325.

20. Szybalski, W. 1985. Universal Restriction Endonucleases: Designing Novel Cleavage Specificities by Combining Adapter Oligodeoxynucleotide and Enzyme Moieties. Gene 40:169-173.

21. Friedman, S. H. and Kenyon, G. L. 1995. Bioorganic Chemistry. In Molecular Biology and Biotechnology: A Comprehensive Desk Reference, R. A. Meyers, Ed., Wiley-VCH, NY, p. 103-106.

We claim:

1. An isolated primer for amplifying a segment of DNA comprising:
a linear oligonucleotide comprising a 5′ end and a 3′ end, said oligonucleotide consisting of at least 35 nucleotides, wherein a first portion of said oligonucleotide of at least 13 nucleotides at the 5′ end of said oligonucleotide and a second portion of the oligonucleotide of from 5 to 22 nucleotides at the 3′ end of the oligonucleotide are precisely complementary to a first portion and a second portion, respectively, of a segment of a cDNA or genomic DNA, wherein 4-8 nucleotides between the first portion and the second portion of the oligonucleotide comprise a recognition site for a restriction endonuclease that cleaves at least 5 nucleotides from its recognition site, wherein the segment of the cDNA or genomic DNA does not comprise the recognition site.

2. The primer of claim 1 wherein the segment of the cDNA or genomic DNA to which the first and second portions of the oligonucleotide are complementary comprises from 0 to 12 nucleotides between the first portion of said segment and the second portion of said segment.

3. The primer of claim 1 wherein the segment of the cDNA or genomic DNA to which the first and second portions of the oligonucleotide are complementary comprises from 4 to 8 nucleotides between the first portion of said segment and the second portion of said segment.

4. The primer of claim 1 wherein the segment of the cDNA or genomic DNA to which the first and second portions of the oligonucleotide are complementary comprises 6 nucleotides between the first portion of said segment and the second portion of said segment.

5. The primer of claim 1 wherein the restriction endonuclease is a Type IIS restriction endonuclease.

6. The primer of claim 5 wherein the Type IIS restriction endonuclease is BpmI.

7. The primer of claim 1 wherein the restriction endonuclease cleaves DNA at least 8 nucleotides from its recognition site.

8. An isolated primer for amplifying a segment of DNA comprising:
a linear oligonucleotide comprising a 5′ end and a 3′ end, said oligonucleotide consisting of at least 35 nucleotides, wherein a first portion of said oligonucleotide of at least 13 nucleotides at the 5′ end of said oligonucleotide and a second portion of the oligonucleotide of from 5 to 22 nucleotides at the 3′ end of the oligonucleotide are substantially complementary to a first portion and a second portion, respectively, of a segment of a cDNA or genomic DNA, wherein 4-8 nucleotides between the first portion and the second portion of the oligonucleotide comprise a recognition site for a restriction endonuclease that cleaves at least 5 nucleotides from its recognition site, wherein the segment of the cDNA or genomic DNA does not comprise the recognition site.

9. The primer of claim 8 wherein the segment of the cDNA or genomic DNA to which the first and second portions of the oligonucleotide are complementary comprises from 0 to 12 nucleotides between the first portion of said segment and the second portion of said segment.

10. The primer of claim 8 wherein the segment of the cDNA or genomic DNA to which the first and second portions of the oligonucleotide are complementary comprises from 4 to 8 nucleotides between the first portion of said segment and the second portion of said segment.

11. The primer of claim 8 wherein the segment of the cDNA or genomic DNA to which the first and second portions of the oligonucleotide are complementary comprises 6 nucleotides between the first portion of said segment and the second portion of said segment.

12. The primer of claim 8 wherein the restriction endonuclease is a Type IIS restriction endonuclease.

13. The primer of claim 12 wherein the Type IIS restriction endonuclease is BpmI.

14. The primer of claim 8 wherein the restriction endonuclease cleaves DNA at least 8 nucleotides from its recognition site.

15. An isolated pair of primers for amplifying a segment of cDNA or genomic DNA,
wherein each primer comprises a linear oligonucleotide comprising a 5′ end and a 3′ end, said oligonucleotide consisting of at least 35 nucleotides, wherein a first portion of said oligonucleotide of at least 13 nucleotides at the 5′ end of said oligonucleotide and a second portion of the oligonucleotide of from 5 to 22 nucleotides at the 3′ end of the oligonucleotide are precisely complementary to a first portion and a second portion of a cDNA or genomic DNA, wherein 4-8 nucleotides between the first portion and the second portion of the oligonucleotide comprise a recognition site for a restriction endonuclease that cleaves at least 5 nucleotides from its recognition site, wherein the segment of the cDNA or genomic DNA does not comprise the recognition site for the restriction endonuclease,
wherein each primer of the pair of primers is complementary to an opposite strand of a double stranded cDNA or genomic DNA molecule, wherein the pair of primers is complementary to two non-contiguous portions of the double stranded cDNA or genomic DNA molecule, wherein 1 to 20 nucleotides separate the two non-contiguous portions of the double stranded cDNA or genomic DNA molecule.

16. The pair of primers of claim 15 wherein the restriction endonuclease is a Type IIS restriction endonuclease.

17. The pair of primers of claim 15 wherein a single nucleotide polymorphism maps to the 1 to 20 nucleotides which separate the two non-contiguous portions of the double stranded DNA molecule.

18. The pair of primers of claim 15 which are contained in a kit.

19. The pair of primers of claim 18 wherein the kit further comprises the restriction endonuclease.

20. A kit comprising a plurality of pairs of primers according to claim 15.

21. An isolated pair of primers for amplifying a segment of cDNA or genomic DNA,
wherein each primer comprises a linear oligonucleotide comprising a 5′ end and a 3′ end, said oligonucleotide consisting of at least 35 nucleotides, wherein a first portion of said oligonucleotide of at least 13 nucleotides at the 5′ end of said oligonucleotide and a second portion of the oligonucleotide of from 5 to 22 nucleotides at the 3′ end of the oligonucleotide are substantially complementary to a first portion and a second portion of a cDNA or genomic DNA, wherein 4-8 nucleotides between the first portion and the second portion of the oligonucleotide comprise a recognition site for a restriction endonuclease that cleaves at least 5 nucleotides from its recognition site, wherein the segment of the cDNA or genomic DNA does not comprise the recognition site for the restriction endonuclease,
wherein each primer of the pair of primers is complementary to an opposite strand of a double stranded cDNA or genomic DNA molecule, wherein the pair of primers is complementary to two non-contiguous portions of the double stranded cDNA or genomic DNA molecule, wherein 1 to 20 nucleotides separate the two non-contiguous portions of the double stranded cDNA or genomic DNA molecule.

22. The pair of primers of claim 21 wherein the restriction endonuclease is a Type IIS restriction endonuclease.

23. The pair of primers of claim 21 wherein a single nucleotide polymorphism maps to the I to 20 nucleotides which separate the two non-contiguous portions of the double stranded DNA molecule.

24. The pair of primers of claim 21 which are contained in a kit.

25. The pair of primers of claim 21 wherein the kit further comprises the restriction endonuclease.

26. A kit comprising a plurality of pairs of primers according to claim 2 1.

27. A method for producing a short segment of DNA, suitable for analysis by mass spectrometry, comprising the steps of:
amplifying cDNA or genomic DNA of a subject using the pair of primers of claim 15 to form amplified DNA;
digesting the amplified DNA with the restriction endonuclease to form a short segment of DNA.

28. A method for producing a short segment of DNA, suitable for analysis by mass spectrometry, comprising the steps of.
amplifying cDNA or genomic DNA of a subject using the pair of primers of claim 21 to form amplified DNA;
digesting the amplified DNA with the restriction endonuclease to form a short segment of DNA.

29. A method for analyzing a first short segment of DNA comprising a first polymorphic nucleotide to distinguish the first short segment of DNA from a second short segment of DNA comprising a second polymorphic nucleotide, the method comprising the steps of:
applying a mixture of DNA segments to an electrospray ionization/mass spectrometer, whereby the DNA segments are denatured and the denatured segments are separated, wherein the mixture of DNA segments is made by the process of:
amplifying cDNA or genomic DNA of a subject using the pair of primers of claim 15 to form amplified DNA; and digesting the amplified DNA with the restriction endonuclease to form a short segment of DNA.

30. A method for analyzing a first short segment of DNA comprising a first polymorphic nucleotide to distinguish the first short segment of DNA from a second short segment of DNA comprising a second polymorphic nucleotide, the method comprising the steps of:
applying a mixture of DNA segments to an electrospray ionization/mass spectrometer, whereby the DNA segments are denatured and the denatured segments are separated, wherein the mixture of DNA segments is made by the process of:
amplifying cDNA or genomic DNA of a subject using the pair of primers of claim 21 to form amplified DNA; anddigesting the amplified DNA with the restriction endonuclease to form a short segment of DNA.


SEQUENCE LISTING (20 sequences)

SEQ ID NO: 1 - DNA, 43 bp, Artificial Sequence
  Description of Artificial SequencePrimer for PCR amplification of human genomic DNA
agacgacacaggaagcagattctggagataccctgcaaatagc

SEQ ID NO: 2 - DNA, 42 bp, Artificial Sequence
  Description of Artificial SequencePrimer for PCR amplification of human genomic DNA
ggaacttcgctcacaggatcttctggagacctagttccaatc

SEQ ID NO: 3 - DNA, 15 bp, Homo sapiens
agaaaaaaaagaaaa

SEQ ID NO: 4 - DNA, 15 bp, Homo sapiens
ttcttttttttctgc

SEQ ID NO: 5 - DNA, 15 bp, Homo sapiens
agaaataaaagaaaa

SEQ ID NO: 6 - DNA, 15 bp, Homo sapiens
ttcttttatttctgc

SEQ ID NO: 7 - DNA, 43 bp, Artificial Sequence
  Description of Artificial SequencePrimer for PCR amplification of human genomic DNA
ttcagagggtccaggttcttcctggagctgatactttattaca

SEQ ID NO: 8 - DNA, 41 bp, Artificial Sequence
  Description of Artificial SequencePrimer for PCR amplification of human genomic DNA
gcactcaggctggatgaacaactggagccatctggagtact

SEQ ID NO: 9 - DNA, 16 bp, Homo sapiens
ttttgccacggaaagt

SEQ ID NO: 10 - DNA, 16 bp, Homo sapiens
tttccgtggcaaaatg

SEQ ID NO: 11 - DNA, 11 bp, Homo sapiens
gcgtcgtcttc

SEQ ID NO: 12 - DNA, 11 bp, Homo sapiens
agacgacgcag

SEQ ID NO: 13 - DNA, 11 bp, Homo sapiens
gcgtcttcttc

SEQ ID NO: 14 - DNA, 11 bp, Homo sapiens
agaagacgcag

SEQ ID NO: 15 - DNA, 43 bp, Artificial Sequence
  Description of Artificial SequencePrimer for PCR amplification of human genomic DNA
ggactacaggccattgcagaactggagcaagtggactgtgaaa

SEQ ID NO: 16 - DNA, 42 bp, Artificial Sequence
  Description of Artificial SequencePrimer for PCR amplification of human genomic DNA
agcatatcgtcttagtgtaatactggagtggtcattagtaag

SEQ ID NO: 17 - DNA, 43 bp, Artificial Sequence
  Description of Artificial SequencePrimer for PCR amplification of human genomic DNA
attttatgtataaattaatctctggaggattaatttgcaggtt

SEQ ID NO: 18 - DNA, 42 bp, Artificial Sequence
  Description of Artificial SequencePrimer for PCR amplification of human genomic DNA
tttactatttacatctgctcgcctggagaaattcctcaaaac

SEQ ID NO: 19 - DNA, 43 bp, Artificial Sequence
  Description of Artificial SequencePrimer for PCR amplification of human genomic DNA
tttccgtgtgaaaaagataatctggagggtccagcaagcatct

SEQ ID NO: 20 - DNA, 42 bp, Artificial Sequence
  Description of Artificial SequencePrimer for PCR amplification of human genomic DNA
ggtttctttttcttaccatctactggagttttgttgggtgca